(19) United States
(12) Patent Application Publication
(10) Pub. No.: US 2012/0156780 A1
(43) Pub. Date: Jun. 21, 2012

(54) Polymer Substrates Having Improved Biological Response From HKDCS

(76) Inventors: Kevin L. Cooper, Flemington, NJ (US); Alisha Martin, Dallas, TX (US); Sriram Natarajan, Bridgewater, NJ (US); Robert Vetrecin, Stewartsville, NJ (US); Changdeng Liu, Belle Mead, NJ (US)

(21) Appl. No.: 12/974,216

(22) Filed: Dec. 21, 2010

(51) Int. Cl. C12N 5/071; C08J 3/28; B32B 3/00

(52) U.S. Cl. 435/396; 428/141; 522/166

(57) ABSTRACT

A method of surface modification of a biocompatible, biodegradable polymer substrate using RF plasma treatment is disclosed. This method and the resulting surface provide for enhanced adhesion and proliferation of cells, such as hKDCs, and can be used with scaffolds for tissue regeneration and with other delivery vehicles such as medical devices.

### FIELD OF THE INVENTION

The invention relates to a method of surface modification of a polymer substrate in order to enhance the biological response of the surface, more specifically biocompatible, biodegradable polymer substrates.

### BACKGROUND OF THE INVENTION

Bioabsorbable polymers are widely used due to their unique attributes for medical devices and pharmaceutical applications including, biocompatibility, strength retention, and sustained release, and also because the bioabsorbable or biodegradable polymer mass is replaced by autologous body tissue. Absorbable polymers are widely used in tissue engineering devices and in substrates to seed and grow cells, but the cells do not always attach, proliferate, and grow well on these devices or substrates. Bioabsorbable polyesters in the polylactide and polyglycolide family are well known and attractive materials for tissue regeneration scaffolds because they have a long and favorable clinical record in various surgical applications and procedures including safety and efficacy, and offer a wide range of physical properties and degradation rates.

Although the specific needs of each tissue are different, many of the same general problems must be solved in adapting bioabsorbable polyesters as scaffolds for wide ranging applications. This includes controlling cell-surface interactions such as adhesion, migration and differentiation. While the situation is complex for amorphous polymers in the family, for example for poly(D,L-lactide) and poly(lactide-co-glycolide) (PLGA), the situation is even more complex for the semi-crystalline polymers, for example for poly(L-lactide) (PLLA) and poly(glycolide) (PGA). It is believed that the degree of crystallinity strongly affects material properties and can also affect cell-surface interactions. The process of tissue regeneration is believed to be governed by the interactions of cells with the surface of a medical device or scaffold. Thus, the surface properties of these materials and how such properties are affected or altered by processing are important to understand and control. Processing these polymers by methods that achieve desired bulk properties may alter surface properties in ways that are not anticipated, and that may have negative effects on desired biological outcomes. It is therefore desirable to develop processes that can affect only the surface of a biodegradable polymeric structure or substrate without altering the bulk properties of the polymer.

Plasma treatment is known and has been used to alter the surface properties of polymers without affecting their bulk properties. Specific surface properties like hydrophobicity, chemical structure, and roughness can be tailored to meet target requirements. Some major effects that have been observed in plasma treatment of polymer surfaces are removal of organic contamination, micro and nano scale-etching, cross-linking and surface chemistry modifications. Plasma techniques for modifying the surface characteristics of many materials are known. Specific applications for surface modified materials have been described for both microelectronic and medical implant device technology.

In the medical device arts, the use of plasma treatment for implantable medical devices made from biocompatible materials has generally been confined to surface conditioning, i.e., altering functional groups on the surface of the devices, without attention to the surface morphology. Descriptions and elaboration of surface modifications for implants and other devices by radio frequency (RF) plasmas can be found in the following U.S. Pat. Nos. 3,814,983; 4,929,319, 4,948,628; 5,055,316; 5,080,924; 5,084,151; 5,217,743; 5,229,172; 5,246,451; 5,260,093; 5,262,097; 5,364,662; 5,451,428; 5,476,509; and 5,543,019.

It has been demonstrated that protein adsorption and endothelial cell attachment, spreading, and proliferation are influenced by both chemical and physical properties of the polymer surface (Lee, J-S. et al., Biomater 14:958-960 (1993)). It has also been shown that endothelial cell proliferation and spreading can be enhanced by increasing the oxygen concentration at the polymer surface (Kottke-Marchant, K. et al. J Biomed Mater Res 30:209-220 (1996); Ertel, S. I. et al. J Biomed Mater Res 24:1637-1659 (1990)). In contrast to ion implantation, plasma surface modification is confined to the outermost surface layer. However, one drawback associated with oxygen and air plasma treatments is the degradation of the material properties as a result of chain scission.

Medical devices that have contact with the human body need an optimal combination of mechanical properties and surface characteristics that result in superior performance in the biological environment. There is then a need in this art for implantable medical devices having modified substrate material surfaces, and methods of producing such surfaces, such that these medical devices have improved performance in biological environments, particularly with respect to promoting desirable cell growth on such surfaces.

### SUMMARY OF THE INVENTION

Accordingly, a novel method of surface modification of a substrate is disclosed. The novel method includes the steps of providing a biocompatible, biodegradable polymer substrate. The substrate has a surface. The polymer is semi-crystalline, and the surface has a crystallinity. The substrate is placed in an inert gas atmosphere. An RF plasma treatment is applied to the surface at a power of from about 100 W to about 500 W for a length of time of about 60 to about 200 minutes, thereby providing a surface crystallinity of about 30 to about 50% and a roughness of from about 20 nm to about 200 nm. The polymer substrate treated in such a manner has improved cell attachment and growth of cells for tissue engineering, including hKDCs, which is important in the area of kidney tissue engineering.

Another aspect of the present invention is a biodegradable polymer substrate having a surface modified by the above-described method.

Yet another aspect of the present invention is a method of growing cells on the above-described substrate.

The foregoing and other features and advantages of the present invention will become more apparent from the following description and accompanying drawings.

### BRIEF DESCRIPTION OF THE DRAWINGS

FIG. 1 is a graph showing the viable cell count analysis on unannealed plasma treated PLLA films.

FIG. 2 is a graph showing the viable cell count analysis on unannealed plasma treated PDO films.

### DETAILED DESCRIPTION OF THE INVENTION

The method of the present invention provides for the altering of surface crystallinity of polymer surfaces, specifically biodegradable, biocompatible polymers, by inert gas plasma treatments, resulting in an enhanced biological response of cells toward the polymer surfaces. The effect is observed with semi-crystalline polymers. The intensity and quality of the plasma to which a target material is exposed produces a randomized, irregularly etched surface that is characterized by dimensional (i.e., depth and width), morphological (i.e., crystallinity) and functional group (i.e. —O, —OH, NH2 etc.) variations on the surface. In order to establish this plasma, low background pressures and relatively low power levels are employed.

The methods of the present invention provide for the surface modification of a substrate by providing a biocompatible, biodegradable, semi-crystalline polymer substrate. The substrate is placed in an inert gas atmosphere and subjected to an RF plasma treatment, thereby producing a surface having increased crystallinity and roughness. The resultant polymer surfaces having increased crystallinity and roughness are shown to surprisingly provide improved cell growth and proliferation for cells, including human kidney-derived cells.

The polymer substrates and medical devices useful in the methods of the present invention are prepared from biocompatible, biodegradable, semi-crystalline polymers. The biodegradable polymers readily break down into small segments when exposed to moist body tissue. The segments then either are absorbed by the body, or passed by the body. More particularly, the biodegraded segments do not elicit permanent chronic foreign body reaction, because they are absorbed by the body or passed from the body, such that no permanent trace or residual of the segment is retained by the body. Biodegradable polymers can also be referred to as bioabsorbable or bioresorbable polymers, and all of these terms can be used interchangeably within the context of the present invention.

Semi-crystalline polymers have both amorphous and crystalline regions. A fraction of the polymer remains un-crystallized, when the polymer is cooled to room temperature. Semi-crystalline polymers typically exhibit crystallinity in the range of from about 10% to about 80%. For the purposes of this invention, the semi-crystalline polymer is able to achieve crystallinity in the range of from about 30% to about 50%. The crystallinity of the bulk polymer may be measured by differential scanning calorimetry while the surface crystallinity may be measured by grazing angle x-ray diffraction, and it will be appreciated by those skilled in the art that other available measurement protocols may be used. All percentages of crystallinity are measured in weight percent unless otherwise noted.

Suitable biocompatible, biodegradable polymers useful in the practice of the present invention include polymers selected from the group consisting of aliphatic polyesters, poly (amino acids), copoly (ether-esters), polyalkylenes oxalates, polyamides, tyrosine derived polycarbonates, poly (iminocarbonates), polyorthoesters, polyoxaesters, polyamidoesters, polyoxaesters containing amine groups, poly (anhydrides), polyphosphazenes, and combinations thereof.

For the purposes of the present invention, aliphatic polyesters include, but are not limited to, homopolymers and copolymers of lactide (which includes lactic acid, D-, L- and meso lactide), glycolide (including glycolic acid), epsilon-caprolactone, p-dioxanone (1,4-dioxan-2-one), trimethylene carbonate (1,3-dioxan-2-one), alkyl derivatives of trimethylene carbonate, and blends thereof.

Suitable biocompatible, biodegradable elastomeric copolymers include but are not limited to copolymers of epsilon-caprolactone and glycolide (preferably having a mole ratio of epsilon-caprolactone to glycolide of from about 30:70 to about 70:30, preferably 35:65 to about 65:35, and more preferably 45:55 to 35:65); elastomeric copolymers of epsilon-caprolactone and lactide, including L-lactide, D-lactide blends thereof or lactic acid copolymers (preferably having a mole ratio of epsilon-caprolactone to lactide of from about 35:65 to about 65:35 and more preferably 45:55 to 30:70) elastomeric copolymers of p-dioxanone (1,4-dioxan-2-one) and lactide including L-lactide, D-lactide and lactic acid (preferably having a mole ratio of p-dioxanone to lactide of from about 40:60 to about 60:40); elastomeric copolymers of epsilon-caprolactone and p-dioxanone (preferably having a mole ratio of epsilon-caprolactone to p-dioxanone of from about 30:70 to about 70:30); elastomeric copolymers of p-dioxanone and trimethylene carbonate (preferably having a mole ratio of p-dioxanone to trimethylene carbonate of from about 30:70 to about 70:30); elastomeric copolymers of trimethylene carbonate and glycolide (preferably having a mole ratio of trimethylene carbonate to glycolide of from about 30:70 to about 70:30); elastomeric copolymer of trimethylene carbonate and lactide including L-lactide, D-lactide, blends thereof or lactic acid copolymers (preferably having a mole ratio of trimethylene carbonate to lactide of from about 30:70 to about 70:30) and blends thereof. In one embodiment, the elastomeric copolymer is a copolymer of glycolide and epsilon-caprolactone. In another embodiment, the elastomeric copolymer is a copolymer of lactide and epsilon-caprolactone.

The substrates or devices treated by the process of the present invention may be of any suitable shape for a medical device or substrate on which it is desired to grow cells, including conventionally known shapes. Suitable cells which may be grown on the modified surfaces include, but are not limited to, stem cells, progenitor cells, primary cells, transfected cells and immortalized cells. In one embodiment the cells are human kidney derived cells. Examples of suitable substrates and devices include, but are non limited to, medical devices, such as suture anchors, sutures, staples, surgical tacks, clips, plates, screws, and films; tissue engineering scaffolds, such as non-woven felts, woven meshes or fabrics; foams; powders; and cell culture vessels, such as, dishes, flasks and the like.

Plasma treatment of the surface of the substrate may be accomplished using cold plasma techniques such as, radio frequency (RF), microwave, direct current (DC), and the like. In one embodiment, the plasma is RF plasma. The plasma treatment is controlled through many variables including, the type of gas, radio frequency, power, duration of treatment, and atmospheric pressure.

The type of gas conventionally used for the RF plasma treatment is typically a reactive gas, such as oxygen, or an inert gas. Typically, reactive gases are used to provide a different chemical composition on the polymer surface. However, the present invention provides an improvement in the growth of cells on an RF plasma treated biodegradable polymer substrate without substantially changing the chemical composition on the surface. In the practice of the present invention, an inert gas is used to physically etch the surface of the substrate and create nano/micro scale textures on the surface. Suitable inert gases include, but are not limited to, nitrogen, argon, and helium.

The RF plasma radio frequency may be up to 100 MHz, preferably in the range of from about 10 MHz to about 45 MHz. In one embodiment, the radio frequency is about 13.56 MHz. In another embodiment, higher radio frequencies in the range of about 30 MHz to about 45 MHz are be used. In general, higher radio frequencies, will increase ion bombardment activity and favor production of more dynamic masking activity, but lower radio frequencies are used to maintain a more uniform plasma. The radiofrequency may also be modulated i.e. the frequency changed during the plasma treatment process. The frequency may be tailored to obtain a plasma with the desired characteristics.

The RF power is sufficient to effectively treat the surface of the substrate, and can typically be between about 5 watts to about 500 watts (W). In one embodiment, the power ranges from about 100 W to 500 W. In another embodiment, the plasma power range may be from 75 W to about 250 W. In yet another embodiment, the power of plasma treatment is about 250 W. The power range will be selected to obtain the desired plasma characteristics.

Optionally, modulation of the RF power level during the plasma treatment can be employed to modify the etching characteristics. Manual and/or programmed rapid and/or slow changes in the amount of radio frequency energy i.e. power being supplied to the plasma are possible. In general, the RF power is set at an initial level, for example 100 watts and subsequently increased and decreased, by for example by 25% from the original power setting, at specified intervals over the course of the etching period. Variations in power will affect the plasma's ability to etch a surface and can increase or decrease its ability to create nano-scale features on the surface.

The plasma treatment pressure will be suffieciently effective to provide the desired treatment, and for example may range from about 0.01 Torr to about 0.50 Torr. In one embodiment, plasma treatment pressure is about 0.03 Torr.

The duration of plasma treatment is a sufficient period of time to provide effective treatment and, for example, may range from about 60 to about 200 minutes. In one embodiment, the duration of plasma treatment is from about 90 to about 100 min.

The bias voltage applied to the sample to be etched and the location of the sample in the plasma chamber and chamber pressure will affect the etching process and ultimately the surface morphology. In one embodiment, polymeric or metallic samples are placed in the center of the plasma on a floating electrode and the chamber pressure is 0.03 Ton. In still other embodiments, the electrode on which the sample is placed is electrically connected to a RF generator or a DC bias is applied.

The plasma chamber or equipment will have a conventional configuration and typically consists of a chamber that has an inlet and an outlet port. The inlet port is used for feeding in the gas of interest. The flow rate is controlled by a mass flow controller. The outlet port is connected to a vacuum pump and is used to evacuate the chamber to remove air and also remove excess gas flowing in. The chamber itself has metallic electrodes through which high voltage can be applied to generate a plasma with the gas of interest.

As a result of the RF plasma treatment, the biocompatible, biodegradable polymer substrate is physically etched on the surface without affecting the bulk substrate properties. By the surface of the substrate, is meant the top layer, in particular the top 50 micron −100 micron layer of the substrate, The RF plasma treatment etches the substrate polymeric surface by removing the amorphous regions while leaving the crystalline regions, thereby increasing the crystallinity of the polymer at the surface of the substrate. Additionally, the RF plasma etching increases the surface roughness of the substrate. By using an inert gas for the RF plasma treatment the surface is physically changed with substantially no change in the chemical composition of the surface. The surface of the substrate typically exhibits crystallinity in the range of from about 30% to about 50% and surface roughness in the range of from about 20 nm to about 100 nm, for example in the case of PLLA. The surface roughness is in the form of numerous sharp peaks and valleys and has improved proliferation and growth of cells on the substrate.

Typically, absorbable polymer films are made by compression molding or extrusion from polymer pellets. The surface roughness and crystallinity of the as prepared films can vary depending on the polymer and method of preparation. In the case of compression molded PLLA films, the crystallinity of the as prepared films is about 3.5% with surface roughness values less than 10 nm.

The RF plasma treated polymer substrates of the present invention are particularly useful for the growth of human kidney-drived cells (hKDCs), although they are useful for other types of cells. Human kidney derived cells are isolated as described in US Patent Publication Number 2008/0112939, hereby incorporated by reference herein in its entirety.

Briefly, human kidney derived cells are isolated from a human kidney, suitable for organ transplantation. Blood and debris are removed from the kidney tissue prior to isolation of the cells by washing with any suitable medium or buffer such as phosphate buffered saline. Human kidney derived cells are then isolated from mammalian kidney tissue by enzymatic digestion. Combinations of collagenase, dispase, and hyaluronidase are used to dissociate cells from the human kidney tissue. Isolated cells are then transferred to sterile tissue culture vessels that are initially coated with gelatin. Human kidney derived cells are cultured in any culture medium capable of sustaining growth of the cells such as, but not limited to, renal epithelial growth medium (REGM).

Human kidney derived cells are passaged to a separate culture vessel containing fresh medium of the same or a different type as that used initially, where the population of cells can be mitotically expanded. The cells of the invention may be used at any point between passage 0 and senescence. The cells preferably are passaged between about 3 and about 20 times, more preferably are passaged about 4 to about 12 times.

In order to deliver the hKDC cells using a biodegradable scaffold, it is necessary to seed the scaffold with cells. In order to be effective, the cells have to adhere to the scaffold and proliferate. Kidney derived cells grown on synthetic, polyester scaffolds to yield tissue-like structures are useful as the basic building block materials for kidney tissue engineering applications. Therefore, it is advantageous to develop substrates that enhance cell adhesion and proliferation of hKDC on biodegradable materials. This substrate with hKDC can be used for tissue engineering and cell culture experiments.

The following examples are illustrative of the principles and practice of the present invention, although not limited thereto.

### EXAMPLES

### Example 1

### Cell Adhesion on Inert Gas Plasma Treated Poly(L-Lactide) (PLLA)

The goal of this experiment was to determine the cell responses to inert gas plasma treated and annealed PLLA films and correlate these to the surface roughness and crystallinity. Polymer films of 150 microns thickness were made from PLLA, obtained from Purac Biomaterials (Birmingham, Ala.), by compression molding 2-3 gram quantities in a at 392° F. for 15 min at 277 psi. Some of the PLLA films were annealed in an oven under nitrogen atmosphere at 110° C. for 12.5 hrs. For plasma treatment, polymer films of approximately 2.5 cm² were placed into the plasma chamber. The plasma chamber was first evacuated of air by a vacuum pump for 15 min and backfilled with inert gas Helium (He) to a pressure of around 35 milliTorr. The plasma was then set to a target power of 100 and 250 W and turned on for a duration of 40 and 90 min respectively. The surface crystallinity of the films was characterized using grazing angle x-ray diffraction (XRD) analysis which was performed at Evans Analytical (Sunnyvale, Calif.).

The cell response of human kidney-derived cells (hKDCs) on the films was evaluated by punching 9.5 mm samples from the films described above and placing them in culture wells using renal basal epithelial medium. The cells were seeded at a concentration of 100,000 cells/punch and incubated at 37° C. overnight. Then the samples were then transferred to a new plate for cell growth and observed at day 2 and day 7 with medium changes every 2-3 days. Cell attachment and proliferation was observed on these films to determine the biological response.

Cells on the polymer substrate were imaged by live-dead staining using the procedure below. The working solution was prepared by diluting the Calcein AM (Live stain) and the Ethidium homodimer (Dead stain) to 2 uM and 4 uM respectively in PBS (stains were combined at the point of use). The media containing the polymer punches was aspirated once with PBS and the Live/Dead stain was added. This was incubated for at least 5 minutes at room temperature then imaged under a microscope. The live cells are seen as green in color and the dead cells are red.

To obtain additional information in some cases, the number of cells was quantified using the Guava cell counter. Media was aspirated from culture well and punches washed with PBS. 0.5 mL 0.25% Trypsin-EDTA was added to each punch and incubated at 37° C./5% CO2 for 5 min and then neutralized with 0.5 mL media. Cell-media suspensions were collected in a micro-centrifuge tube and tubes were centrifuged for 5 mins/5000 rpm. Cell pellets were resuspended in 0.5 mL (PBS/0.3% FBS). 0.150 mL cell suspension was transferred to a 96 well plate with each sample evaluated in triplicate. 0.050 mL Via Flex dye sol/well was added and samples analyzed on Guava instrument. 3 data points/punch was obtained and averaged to give the cell count

### Results:

The surface crystallinity and values are shown in Table 1.

TABLE 1
Surface Crystallinity of Helium Plasma Treated PLLA Samples

| | Surface Crystallinity from |
|---|---|
| | grazing XRD (%) |
| Annealed - No | 45.6 |
| plasma treatment | |
| Unannealed - No | 3.5 |
| plasma treatment | |
| Annealed - He | 48.8 |
| Plasma treatment - | |
| 250 W, 90 min | |
| Unannealed - He | 44.3 |
| plasma treatment - | |
| 250 W, 90 min | |
| Annealed - He | 44.7 |
| Plasma treatment, | |
| 100 W, 40 min | |
| Unannealed - He | 1.6 |
| plasma treatment, | |
| 100 W, 40 min | |

The XRD measurements indicate that the annealing process increased the crystallinity of the film. Since annealing is a bulk process, the crystallinity of the bulk film was also increased by this process. These results further show that the He plasma treatment enhanced the surface crystallinity of the unannealed films (from 3.5 to 44.3) while the crystallinity of the annealed film changed very little (from 45.6 to 48.8). It is postulated that the surface crystallinity of the unannealed film changes due to preferential etching of the amorphous regions by the inert gas He.

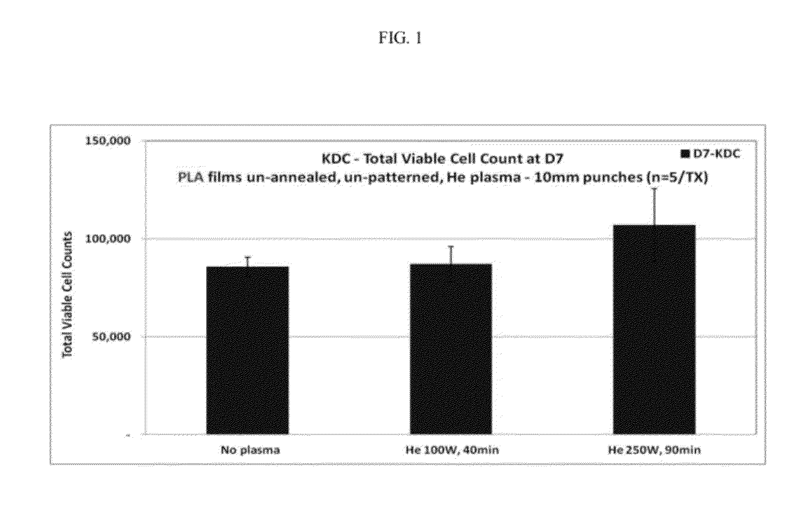

The cell counts measured for the plasma treatments are shown in FIG. 1. This graph shows that maximum hKDC attachment was on the 250 W, 90 min Helium plasma treated un-annealed PLLA. If a lower intensity He plasma is used (100 W, 40 min) the change in crystallinity compared to untreated PLLA was much less (Table 1) and so there was not much change in the cell response. The images from the live-dead staining also show maximum cell density on the 250 W, 90 min unannealed plasma treated PLLA sample. Even though the crystallinity of the annealed PLLA sample (both untreated and plasma treated) was high (about 45 to 49 percent) the images from the live-dead staining did not show as much cell attachment as the 250 W, 90 min unannealed plasma treated PLLA sample. This means that increased surface crystallinity appeared to be more important for cell attachment and this can be achieved by the inert gas plasma treatment described in this example.

### Example 2

### Cell Adhesion on Reactive Gas Plasma Treated Poly-Lactide (PLLA)

The goal of this experiment was to determine the cell response to reactive gas plasma treated and annealed PLLA films and correlate these to the surface roughness and crystallinity. Polymers films were made and annealed as described in Example 1. For plasma treatment, polymer films of approximately 2.5 cm² were placed into the plasma chamber. The plasma chamber was first evacuated of air by a vacuum pump for 15 min and backfilled with reactive gas oxygen to a pressure of around 30 milliTorr. The plasma was then set to a target power of 100 W and turned on for duration of 10 min. XRD analysis, cell seeding and evaluation of cell growth and attachment were performed as described in Example 1.

### Results:

TABLE 2
Surface Crystallinity of Oxygen Plasma Treated PLLA Samples

| | Surface Crystallinity from |
|---|---|
| | grazing XRD (%) |
| Annealed - No plasma | 45.6 |
| treatment | |
| Unannealed - No plasma | 3.5 |
| treatment | |
| Annealed - Oxygen | 50.6 |
| Plasma treatment - 100 W, | |
| 10 min | |
| Unannealed - Oxygen | 14.6 |
| plasma treatment - 100 W | |
| 10 min | |

The XRD measurements in Table 2 show that the oxygen plasma treatment does not enhance the surface crystallinity of the unannealed films as much as the He plasma treatment, while the crystallinity of the annealed film changes very little (from 45.6 to 50.6). Since oxygen is a very reactive gas, increasing the time or power of plasma treatment will lead to the absorbable polymers being aggressively reacted away. Hence the time and power of treatment is limited and this cannot change the surface crystallinity values by much.

The images from the live-dead staining show sparse cell attachment on the oxygen plasma treated PLLA samples. Therefore, desired surface crystallinity that is important for cell attachment cannot be achieved by the reactive gas plasma treatment described in this example.

### Example 3

### Cell Adhesion on Plasma Treated Polydioxanone (PDO)

The goal of this experiment was to determine the cell response to plasma treated and annealed PDO films and correlate these to crystallinity. Polymer films of 150 microns thickness were made from PDO, obtained from Purac Biomaterials (Birmingham, Ala.), by compression molding 2-3 gram quantities at 293° F. for 10 min at 277 psi. Some of the PDO films were annealed in an oven under nitrogen atmosphere at 70° C. for 12.5 hrs. For plasma treatment, polymer films of approximately 2.5 cm² were placed into the plasma chamber. The plasma chamber was first evacuated of air by a vacuum pump for 15 min and backfilled with reactive gas oxygen or inert gas helium to a pressure of around 30 milliTorr for oxygen and 35 milliTorr for helium. The plasma was then set to a target power of 100 W and 250 W and turned on for a duration of 10 min, 40 min and 90 min for Oxygen gas and Helium gas respectively. Grazing angle XRD analysis was performed at Evans Analytical (Sunnyvale, Calif.) to characterize the surface crystallinity of these films.

The cell response of human kidney-derived cells (hKDCs) on the films was evaluated by punching 9.5 mm samples from the films described above and placing them in culture wells with renal basal epithelial medium. The cells were seeded at a concentration of 100,000 cells/punch and incubated at 37° C. overnight. Then the samples were then transferred to a new plate for cell growth and observed at day 2 and day 7 with the culture medium changed every 2-3 days.

Cell attachment and proliferation was observed on these films using the live dead technique and cell counting techniques described in Example 1.

### Results:

The XRD measurements in Table 3 show that the surface crystallinity of both the unannealed and annealed untreated PDO films is around 30% and did not change significantly, even after the highest plasma treatment conditions.

TABLE 3
Surface Crystallinity of Plasma Treated PDO Samples

| | Surface Crystallinity |
|---|---|
| | from grazing XRD (%) |
| Annealed - No | 30.5 |
| plasma treatment | |
| Unannealed - No | 33.2 |
| plasma treatment | |
| Annealed - He | 41.9 |
| Plasma treatment, | |
| 250 W, 90 min | |
| Unannealed - He | 38.2 |
| plasma treatment, | |
| 250 W, 90 min | |

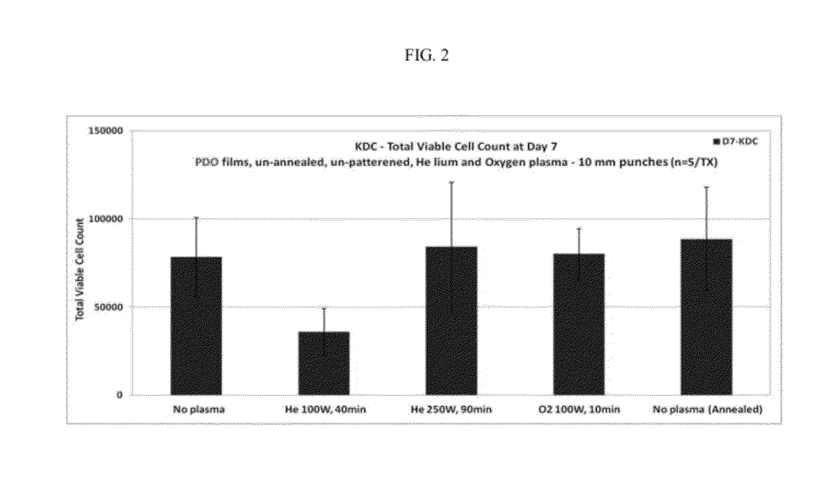

The cell counts measured for the plasma treatments are shown in FIG. 2. There was not much change in cell attachment and proliferation between the various treatment conditions and there was a decrease in the cell attachment for the low power Helium treatment. Since surface crystallinity did not change much, cell attachment and proliferation did not change much.

The above descriptions are merely illustrative and should not be construed to capture all consideration in decisions regarding the optimization of the design and material orientation. It is important to note that although specific configurations are illustrated and described, the principles described are equally applicable to many already known stent configurations. Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope for the appended claims.

We claim:

1. A method of surface modification of a substrate, comprising the steps of:
providing a biocompatible, biodegradable polymer substrate, said substrate having a surface, wherein said polymer is semi-crystalline, and wherein said surface has a crystallinity;
placing the substrate in an inert gas atmosphere;
applying an RF plasma treatment at a power of from about 100 W to about 500 W for a length of time of about 60 to about 200 minutes,
thereby providing the substrate with a surface crystallinity of about 30 to about 50% and a roughness of from about 20 nm to about 200 nm.

2. The method of claim 1, where the substrate comprises a biocompatible, biodegradable aliphatic polyester polymer.

3. The method of claim 2, wherein the aliphatic polyester polymer is selected from the group consisting of homopolymers and copolymers of lactide, glycolide, epsilon-caprolactone, p-dioxanone, trimethylene carbonate, alkyl derivatives of trimethylene carbonate, and combinations thereof.

4. The method claim 3, where the alphatic polyester polymer is poly(L-lactide).

5. The method of claim 1, where the inert gas is selected from the group consisting of nitrogen, argon, and helium.

6. The method of claim 1, where RF power ranges from about 100 W to about 500 W for a length of time from about 60 minutes to about 200 minutes.

7. The method of claim 1, wherein the substrate comprises a medical device.

8. The method of claim 1, wherein the substrate comprises a tissue engineering scaffold.

9. A surface-modified substrate made by the method of claim 1.

10. A substrate comprising a biocompatible, biodegradable polymer, said substrate having a surface, wherein the surface has a crystallinity in the range of about 30% to about 50% and a surface roughness in the range of about 20 nm to about 100 nm.

11. A method of growing a cell on the substrate of claim 10, wherein the cell is selected from the group consisting of human kidney derived cells, stem cells, progenitor cells, primary cells, transfected cells and immortalized cells.

12. The method of claim 11, wherein the substrate comprises a semi-crystalline biodegradable polymer

13. The method of claim 11, wherein the cell comprises a human kidney derived cell.

14. The substrate of claim 10, wherein the biodegradable polymer is selected from the group consisting of homopolymers and copolymers of lactide, glycolide, epsilon-caprolactone, p-dioxanone, trimethylene carbonate, alkyl derivatives of trimethylene carbonate, and combinations thereof.

15. The substrate of claim 10, wherein the substrate comprises a medical device.

16. The substrate of claim 10 wherein the substrate comprises a tissue engineering scaffold.
